(19) United States
(12) Patent Application Publication
(10) Pub. No.: US 2024/0318131 A1
(43) Pub. Date: Sep. 26, 2024

(54) AGROBACTERIUM FOR REDUCING UPTAKE OF HEAVY METALS BY WHEAT AND USE THEREOF

(71) Applicant: Anhui Agricultural University, Hefei (CN)

(72) Inventors: Leni SUN, Hefei (CN); Jie ZHUANG, Hefei (CN); Ruibo SUN, Hefei (CN); Yuanyuan CAO, Hefei (CN); Endong YANG, Hefei (CN); Xiaolin CHEN, Hefei (CN)

(73) Assignee: Anhui Agricultural University, Hefei (CN)

(21) Appl. No.: 18/590,885

(22) Filed: Feb. 28, 2024

(30) Foreign Application Priority Data
Mar. 1, 2023 (CN) 202310201919.6

(51) Int. Cl. C12N 1/20; B09C 1/10; C12R 1/01

(52) U.S. Cl. CPC C12N 1/205; B09C 1/10; B09C 2101/00; C12R 2001/01

(57) ABSTRACT

The present disclosure relates to the technical fields of agriculture and environmental microbiology, and provides Agrobacterium for reducing uptake of heavy metals by wheat. The Agrobacterium is strain NJ20, which is deposited in China General Microbiological Culture Collection Center under Accession CGMCC No. 26197. The present disclosure also provides a bacterial agent which is prepared by the aforementioned Agrobacterium. The Agrobacterium and bacterial agent thereof are used for reducing uptake of heavy metals in heavy metal contaminated soil by wheat. The strain NJ20 of the present disclosure can grow well under the stress of lead and cadmium, and remove soluble lead and cadmium in the culture solution by adsorption; meanwhile the strain NJ20 can generate indoleacetic acid, dissolve insoluble tricalcium phosphate and improve phosphorus nutrition in soil. Inoculation of the strain NJ20 into heavy metal contaminated soil can reduce the contents of lead and cadmium in wheat grains and straws.

### CROSS-REFERENCE TO RELATED APPLICATIONS

The application claims priority to Chinese Patent Application No. 202310201919.6, filed on Mar. 1, 2023, which is incorporated herein by reference in its entirety.

### TECHNICAL FIELD

The present disclosure relates to the technical fields of agriculture and environmental microbiology, particularly to Agrobacterium for reducing uptake of heavy metals by wheat, and use thereof.

### BACKGROUND

With the extensive exploitation of mineral resources around the farmlands, the abuse of pesticides and the development of industrialization, there is a phenomenon of excessive heavy metal content in a large number of farmland soils. Different from organic contamination, heavy metals cannot be degraded and increasingly accumulated in the environment over time. Heavy metal contamination can change the compositions, diversity and biological activity of microflora in soil and the biomass of plants, leading to the loss of soil fertility. The heavy metals in soil can be absorbed by plants and accumulated at edible parts of plants, which not only reduces the quality of agricultural products, but also can be transmitted to a human body through food chains, harming human health. Blocking the accumulation of heavy metals in plants using rhizospheric microorganisms has become one of research hotspots. Compared with the conventional physical and chemical passivator, the rhizospheric microorganisms have the characteristics of low price, convenient operation and improvement of soil ecological environments.

Microbial strains reducing uptake and accumulation of heavy metals by plants, which have been found at present, mainly include Pseudomonas, Bacillus, Enterobacter, Serratia and the like. The microorganisms exert interaction in a soil-microorganism-plant complex system, and are also affected by many factors such as soil and heavy metal concentrations. Screening stable, multifunctional and wide-adaptability heavy metal immobilizing bacteria is of great significance for reducing uptake and transport of heavy metals by plants and improving food safety.

### SUMMARY

The technical problem to be solved by the present disclosure is to provide Agrobacterium for reducing uptake of heavy metals by wheat, and use thereof.

In the present disclosure, the aforementioned technical problem is solved by using the following technical solution:

Provided is Agrobacterium for reducing uptake of heavy metals by wheat, where the Agrobacterium is strain NJ20, which is identified as Agrobacterium sp. NJ20, deposited in China General Microbiological Culture Collection Center of Beijing under Accession CGMCC No. 26197 on Dec. 14, 2022, and exhibits survival.

The bacterial colony of the strain NJ20 is round and white, and has neat edges on the LB culture medium. The strain is negative to Gram staining, negative to amylase, positive to V.P, positive to M-R, positive to catalase, and positive to oxidase. The strain is tolerant to 2% sodium chloride, and grows well at 35° C.

As one of preferred embodiments of the present disclosure, the 16S rDNA sequence of the strain NJ20 is as set forth in SEQ ID NO.1.

As one of preferred embodiments of the present disclosure, the strain NJ20 is used for reducing uptake of lead and cadmium by wheat.

A use of the aforementioned Agrobacterium for reducing uptake of heavy metals by wheat in reducing uptake of lead and cadmium in heavy metal contaminated soil by wheat.

Provided is a bacterial agent prepared by using the aforementioned Agrobacterium for reducing uptake of heavy metals by wheat.

As one of preferred embodiments of the present disclosure, the bacterial agent is a bioremediation liquid bacterial agent containing strain NJ20 whose effective viable count is greater than 200 million CFU/milliliter.

A use of the aforementioned bacterial agent in reducing uptake of heavy metals by wheat in heavy metal contaminated soil.

Compared with the prior art, the present disclosure has the following advantages:
(1) The present disclosure screens Agrobacterium sp. NJ20 capable of tolerating high cadmium and lead, this strain can grow well in a culture solution containing cadmium or lead, and remove soluble cadmium or lead in the culture solution by adsorption, with cadmium and lead removal rates being up to 55.0% and 55.11% respectively.
(2) The Agrobacterium sp. NJ20 can generate indoleacetic acid, which has a good dissolution effect on insoluble tricalcium phosphate, with a phosphate solubilization capacity of up to 190.57 mg/L.
(3) The Agrobacterium sp. NJ20 can simultaneously reduce the contents of cadmium and lead in aboveground tissues (straw and grains) of wheat; Agrobacterium sp. NJ20 is inoculated into two heavy metals contaminated soil so that the contents of cadmium in wheat roots, straws and grains are significantly reduced by 20.55%-29.76%, 38.70%-41.54% and 26.17%-38.86%, respectively; the contents of lead are decreased by 2.15%-4.28%, 64.51%-68.30% and 45.40%-53.02%, respectively; the bacterial agent prepared by the strain provided by the present disclosure can reduce the contents of lead and cadmium in wheat aboveground parts (straw and grains), and is used for reducing uptake and accumulation of lead and cadmium in composite contaminated soil, which is beneficial for safe production of wheat.
(4) The inoculation of Agrobacterium sp. NJ20 can improve the contents of organic matters and available phosphorus in wheat rhizosphere soil, increase the fertility of soil and improve the ecological environment of soil.

### BRIEF DESCRIPTION OF THE DRAWINGS

FIG. 1 is a normal growth bacterial colony chart (without addition of any heavy metals) of strain NJ20 in example 3;

FIG. 2 is a bacterial colony chart of strain NJ20 growing on an agar plate containing 100 mg/L Cd²⁺ in example 3;

FIG. 3 is a bacterial colony chart of strain NJ20 growing on an agar plate containing 150 mg/L Cd²⁺ in example 3;

FIG. 4 is a bacterial colony chart of strain NJ20 growing on an agar plate containing 1400 mg/L Pb²⁺ in example 3.

### DETAILED DESCRIPTION OF THE EMBODIMENTS

In the following, the embodiments of the present disclosure will be illustrated in detail. The embodiments will be implemented on the premise of the technical solution of the present disclosure, and give detailed implementations and specific operation process, however, the scope of protection of the present disclosure is not limited to the following examples.

### Example 1: Isolation of Strain

The isolated and deposited Agrobacterium sp. NJ20 in the present disclosure was obtained through purification and isolation from wheat rhizosphere soil. The specific isolation steps were as follows:

Vigorously grown wheat plants with roots and soil were dig out, the roots were shaken to remove non-rhizosphere soil far from the roots, and the rhizosphere soil attached to the root surface within 1-2 mm was carefully collected using a sterile brush. 1 g of wheat rhizosphere soil was weighed and added into 100 mL of sterilized sterile water, and then the above soil was placed on a table concentrator to be vibrated for 30 min at 28° C. under 180 r/min so as to obtain a 10⁻² soil suspension. 1 mL of 10⁻² soil suspension was sucked and added into 9 mL of sterile water to be evenly mixed to obtain a 10⁻³ soil diluted solution, and then the above 10⁻³ soil diluted solution was diluted by 10 folds in sequence, with the same operations as the above, so as to obtain a 10⁻⁴ soil diluted solution and a 10⁻⁵ soil diluted solution. 100 μL of diluted solution was sucked and coated onto an LB solid culture medium (10.0 g of peptone, 5.0 g of yeast powder, 10.0 g of sodium chloride, 20.0 g of agar, 1000 mL of distilled water, pH7.0-7.2) plate containing Cd²⁺25 mg/L (CdCl₂), and cultured for 48 h in a biochemical incubator at 28-30° C., vigorously grown bacterial colonies were picked after 48 h and streaked on a new LB solid culture medium plate containing Cd²⁺25 mg/L (CdCl₂) and then cultured for 48 h at 28° C., the above operation was repeated 3 times, a single bacterial colony was picked and added into the LB culture medium and placed on the table concentrator to be cultured for 28-36 h at 28° C. under 180 r/min, and then the strain is deposited at −80° C. using 40% glycerol suspension for later use.

### Example 2: Identification of Strain NJ20

The strain NJ20 was detected according to Handbook for Identification of Common Bacterial Systems.

When the strain NJ20 grew in LB culture medium, the bacterial colony was white and raised, had neat edges and a smooth and moist surface, and was opaque. The physiological and biochemical characteristics of the bacteria: it was negative to Gram staining, negative to amylase, positive to V.P, positive to M-R, positive to catalase, positive to oxidase and tolerant to 2% sodium chloride, and grew well at 35° C.

The genome DNA of the strain NJ20 was extracted, and then polymerase chain reaction (PCR) amplification was performed on DNA using general primers 27F and 1492R of bacterial 16S rDNA. The obtained PCR amplification product was sequenced. BLAST analysis was performed using NCBI nucleic acid database according to bacterial 16S rDNA sequence. The analysis results show that the strain NJ20 belongs to the genus Agrobacterium. The results of combining with Gram staining and other physiological and biochemical tests show that the bacterium is Agrobacterium, the strain NJ20 was named as Agrobacterium sp. NJ20. The strain NJ20 was deposited in China General Microbiological Culture Collection Center under Accession CGMCC No. 26197 on Dec. 14, 2022.

The 16S rDNA sequence of the strain Agrobacterium sp. NJ20 is as set forth in SEQ ID NO.1.

### Example 3: Tolerance of Strain NJ20 on Lead and Cadmium

Heavy metal tolerance test was performed on the screened strain NJ20.

LB plates containing different concentration gradients of Cd²⁺ (50, 100, 120, 150, 200 mg/L) and Pb²⁺ (200, 600, 1000, 1200, 1400, 1500 mg/L) were prepared. The strain were streaked and inoculated into solid plate containing heavy metals, and cultured for 4 d at 28° C., and then the growth of the strain was observed.

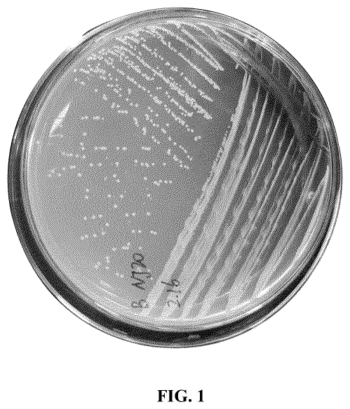

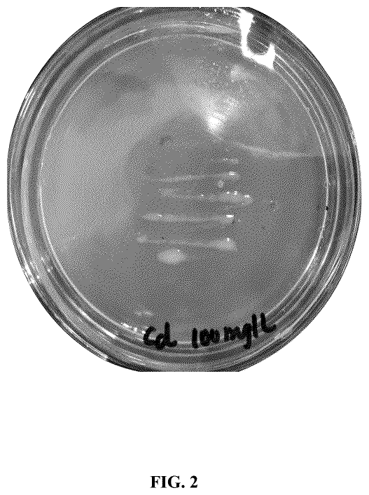

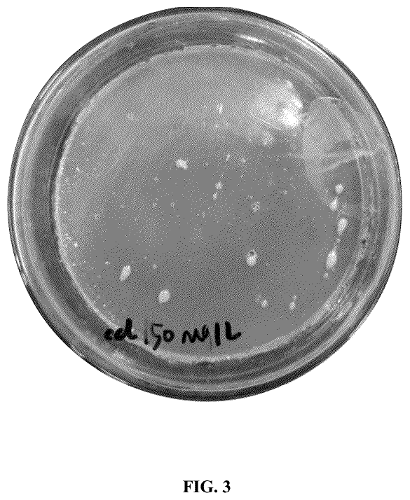

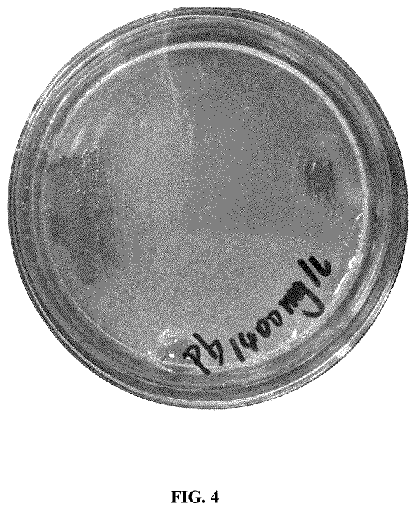

The results show that there are strain NJ20 bacterial colonies on the plate containing 50, 100, 120 and 150 mg/L Cd²⁺; however, as the concentration of Cd²⁺ increases, the growth of the bacterial colonies is becoming increasingly weak, no bacterial colonies occur on the plate any more when the concentration of Cd²⁺ reaches 200 mg/L, indicating that the lethal concentration of Cd²⁺ is 200 mg/L. There are strain NJ20 bacterial colonies on the plates containing 200, 600, 1000, 1200 and 1400 mg/L Pb²⁺, similarly, as the concentration of Ph²⁺ increases, the growth of the bacterial colonies is gradually weakened, no bacterial colonies occur on the plate any more when the concentration of Pb²⁺ reaches 1500 mg/L, indicating that the lethal concentration of Pb²⁺ is 1500 mg/L (refer to FIG. 1-FIG. 4).

### Example 4: Determination of Ability of Strain NJ20 to Remove Lead and Cadmium

The single bacterial colony of strain NJ20 was picked and transferred into the LB liquid culture medium, and then subjected to shaking culture on a table concentrator at 28-30° C. under 180 r/min for 30-40 h, and the culture solution was inoculated into 100 mL of LB liquid culture medium containing 5 mg/L Cd²⁺ or 20 mg/L lead with an inoculation amount of 5% and subsequently subjected to shaking culture on the table concentrator at 28° C. under 180 r/min for 72 h. After culture, supernatant was collected at 6000 r/min, and the contents of heavy metals were measured using atomic absorption spectroscopy. The results are seen in Table 1.

It can be seen from Table 1 that the average cadmium removal rate of strain NJ20 is 55.0%, and the average lead removal rate of strain NJ20 is 55.11%.

TABLE 1
Removal effects of strain NJ20 on cadmium
and lead in culture solution

| | Concentration | | Concentration | |
|---|---|---|---|---|
| | of cadmium in | Cadmium | of lead in | Lead |
| | supernatant | removal | supernatant | removal |
| Strain | mg/L | rate | mg/L | rate |
| NJ20 | 2.25 | 55.0% | 8.75 | 55.11% |

### Example 5: Determination of Abilities of Strain NJ20 to Solubilize Phosphorus and Produce Indoleacetic Acid (IAA)

### 1. The Ability of Strain NJ20 to Solubilize Phosphorus

The strain is inoculated into a 5 mL tube and subjected to shaking culture on a table concentrator for 24-30 h, then inoculated into 100 mL of phosphorus solubilizing culture medium (10.0 g of glucose, 0.1 g of ammonium sulfate, 0.2 g of potassium chloride, 0.25 g of magnesium sulfate heptahydrate, 5.0 g of magnesium chloride hexahydrate, 5.0 g of calcium phosphate, 1000 mL of distilled water, pH 7.0, 115° C., 30 min) with an inoculation amount of 5% and cultured for 3 days on the table concentrator at 28-30° C. under 160 r/min. The culture solution was centrifuged at 8000 r/min, 20 μL of supernatant was taken and 1 mL of molybdenum antimony anti-chromogenic agent was added, distilled water was added until a constant volume reached 10 mL, and an OD value at 700 nm was measured after 15-20 min of development. A 5 mg/L phosphorus standard solution was subjected to gradient dilution until the contents of phosphorus were 0.00, 0.25, 0.50, 1.00, 1.25 and 1.5 mg/L, and then a standard curve was plotted. At room temperature, the molybdenum antimony anti-chromogenic agent was added for 15-20 min of development, and then its light absorption values were measured and a standard curve was plotted. The standard curve was measured as y=0.4721x+0.0144, with a correlation coefficient R²=0.9964. Depending on the standard curve, the content of phosphorus in the strain NJ20 culture solution was calculated as 190.57 mg/L. As can be seen, the strain has a strong phosphorus solubilizing ability, i.e., it can transform insoluble calcium phosphate into soluble phosphorus.

### 2. Determination of Indoleacetic Acid (IAA) Secretion Volume of Strain NJ20

The standard curve was plotted using analytically pure IAA, a 0.2 mg/mL IAA standard solution was subjected to gradient dilution until the concentrations of 0, 5, 10, 15, 20, 25 and 30 mg/L were reached, and the light absorption value (OD530) was measured using Salkowski's chromogenic method. The obtained standard curve was y=0.0316x+0.0112, with a correlation coefficient R²=0.9973.

A nitrogen-containing culture medium (10.0 g of sucrose, 1.0 g of ammonium sulfate, 2.0 g of dipotassium hydrogen phosphate, 0.5 g of magnesium sulfate heptahydrate, 0.1 g of sodium chloride, 0.5 g of yeast extract, 0.5 g of calcium carbonate, pH 7.2, 1000 mL of distilled water, 115° C., 30 min) was sub-packaged into tubes with 4 mL in each tube, 1 mL of tryptophan that was sterilized by filtration was added after sterilization so that the concentration of tryptophan in the culture medium was 0.5 mg/mL, target strains were inoculated into the culture medium and cultured for 2d on a table concentrator at 28° C. under 150 r/min, the culture solution was centrifuged for 10 min at 6000 r/min, 1 mL of supernatant was taken, 50 μL of 10 mmol/L orthophosphoric acid was added and then 2 mL of Sackowski's chromogenic reagent was added, and the above materials were sufficiently mixed and then developed for 30 min at 25° C. in the dark. The absorbance at 530 nm was measured, and the content of IAA added into the strain NJ20 culture solution was calculated as 12.51 mg/L depending on the standard curve.

### Example 6: Activation of Strain NJ20 and Preparation of Bacterial Suspension

Strain NJ20 was picked from an NJ20 glycerinum tube using an inoculation ring, streaked on LB solid culture medium under the sterile operation and cultured for 72 h at 28° C. Then, the single bacterial colony of vigorously grown strain NJ20 was selected and inoculated in LB liquid culture medium and subjected to shaking culture for 20-30 h on a table concentrator at 28° C. under 150-180 r/min.

100 mL of culture solution containing strain NJ20 was taken and centrifuged at 8000 r/min, bacterial cells were collected and washed with sterile water, then bacterial cells were re-suspended in 80-100 mL of sterile water so that the quantity of cells reached 200 million CFU/mL or more, so as to obtain a strain NJ20 liquid bacterial agent.

### Example 7: Blocking Effect of Strain NJ20 on Uptake of Heavy Metals by Wheat

This example was conducted using a pot experiment, and test soils were H soil and F soil.

The two soils (H soil and F soil) were respectively collected from heavy metal contaminated farmlands in different mining areas of Lujiang County, Hefei City. The two soils (H soil and F soil) were respectively sieved by a 100-mesh sieve, 3.5 kg of soil for each pot. Where, the physical and chemical properties of the H soil were as follows: 960 mg/kg of total lead, 5.10 mg/kg of total cadmium, 41.95 mg/kg of available lead, 0.61 mg/kg of available cadmium, 6.17 g/kg of organic matter and 66.09 mg/kg of available phosphorus. The physical and chemical properties of the F soil were as follows: 191 mg/kg of total lead, 2.96 mg/kg of total cadmium, 30.86 mg/kg of available lead, 0.13 mg/kg of available cadmium, 6.17 g/kg of organic matter and 34.79 mg/kg of available phosphorus.

The full and uniformly sized wheat seeds were selected and surface disinfected with 75% ethanol for 3 min, washed many times with plenty of sterile water, and then wrapped with sterile moist gauze for germination treatment. After the seeds germinated, they were soaked for 60-120 min using “strain NJ20 liquid bacterial agent” prepared in example 6. The wheat seeds in control group were soaked with sterile water for the same period of time. 4 treatment groups were set for test, with 4 repetition times in each group. The treatment of 4 groups was specifically as follows:
Treatment group 1: non-inoculation treatment for H soil (H-CK);
Treatment group 2: inoculation treatment for H soil (H-NJ20);
Treatment group 3: non-inoculation treatment for F soil (F-CK);
Treatment group 4: inoculation treatment for F soil (F-NJ20).

12 soaked wheat seeds were planted in soil per pot, and 15 mL of bacterial suspension was applied around the seeds. After 3 weeks of wheat emergence, seedlings were thinned out to retain 9 seedlings per pot. And 10 mL of bacterial suspension was added by watering the roots. After wheat was matured, the grains, straws and roots of the wheat were collected and dried, and then the contents of lead and cadmium were measured.

Measurement method: the grains, straws and roots of the wheat were respectively ground into powders, and measurement was performed using nitric acid-perchloric acid-atomic absorption spectroscopy. And the transport coefficient and enrichment coefficient of the wheat on lead and cadmium were calculated. The transport coefficient=the content of heavy metals in grains or straws/content of heavy metals in the roots; the enrichment coefficient=the content of heavy metals in roots/the content of heavy metals in soil. The results are seen on Table 2, Table 3, Table 4 and Table 5.

TABLE 2
Effect of strain NJ20 on content of cadmium in each part of wheat (mg/kg)

| | Treatment | Roots | Straws | Grains |
|---|---|---|---|---|
| H soil | Control | 3.684 ± 0.008 | 1.613 ± 0.87 | 1.154 ± 0.011 |
| | group | | | |
| | NJ20 | 2.927 ± 0.045** | 0.943 ± 0.059*** | 0.852 ± 0.082*** |
| F soil | Control | 0.504 ± 0.007 | 0.261 ± 0.006 | 0.175 ± 0.004 |
| | group | | | |
| | NJ20 | 0.354 ± 0.002** | 0.160 ± 0.002** | 0.107 ± 0.001** |

TABLE 3
Effect of strain NJ20 on content of lead in each part of wheat (mg/kg)

| | Treatment | Roots | Straws | Grains |
|---|---|---|---|---|
| H soil | Control | 52.076 ± 0.648 | 2.136 ± 0.170 | 0.696 ± 0.145 |
| | group | | | |
| | NJ20 | 50.957 ± 0.554 | 0.758 ± 0.178*** | 0.327 ± 0.025* |
| F soil | Control | 24.680 ± 0.070 | 1.202 ± 0.106 | 0.478 ± 0.021 |
| | group | | | |
| | NJ20 | 23.623 ± 0.315** | 0.381 ± 0.024** | 0.261 ± 0.044* |

Note: * represents p<0.05, ** represents p<0.01, *** represents p<0.001, indicating that there is significant difference between treatments.

It can be seen from Table 2 and Table 3 that in the H soil, the content of cadmium in roots in inoculation treatment group (H-NJ20) is significantly reduced by 20.55%, and the content of lead in roots in inoculation treatment group (H-NJ20) is reduced by 2.15%, as compared with those in control group (H-CK); the content of cadmium in grains is significantly reduced by 26.17%, and the content of lead in grains is significantly reduced by 53.02%, as compared with those in control group (H-CK); the content of cadmium in straws is significantly reduced by 41.54%, and the content of lead in straws is significantly reduced by 64.51%, as compared with those in control group (H-CK).

In the F soil, the content of cadmium in roots in inoculation treatment group (F-NJ20) is significantly reduced by 29.76%, and the content of lead in roots in inoculation treatment group (F-NJ20) is significantly reduced by 4.28%, as compared with those in control group (F-CK); the content of cadmium in grains is significantly reduced by 38.86%, and the content of lead in grains is significantly reduced by 45.40%, as compared with those in control group (F-CK); the content of cadmium in straws is significantly reduced by 38.70%, and the content of lead in straws is significantly reduced by 68.30%, as compared with those in control group (F-CK). Accordingly, except the content of lead in roots, inoculation treatment can significantly reduce the content of cadmium and lead in each tissue (roots, straws and grains) of wheat.

TABLE 4
Effect of strain NJ20 on transport coefficients of lead and cadmium in wheat

| | Transport coefficient | | Transport coefficient | |
|---|---|---|---|---|
| | TF_{grain/root} | | TF_{straw/root} | |
| Treatment | Cd | Pb | Cd | Pb |
| H-CK | 0.316 ± 0.001 | 0.012 ± 0.000 | 0.451 ± 0.009 | 0.039 ± 0.001 |
| H-NJ20 | 0.307 ± 0.008 | 0.007 ± 0.000* | 0.333 ± 0.008* | 0.013 ± 0.001* |
| F-CK | 0.165 ± 0.008 | 0.019 ± 0.001 | 0.518 ± 0.012 | 0.049 ± 0.004 |
| F-NJ20 | 0.158 ± 0.001 | 0.011 ± 0.002* | 0.450 ± 0.005* | 0.016 ± 0.001* |

Note:
*represents that there is significant difference between treatment (p < 0.05).

TABLE 5
Effect of strain NJ20 on enrichment coefficients
of lead and cadmium in wheat

| | | Cadmium enrichment | Lead enrichment |
|---|---|---|---|
| | Treatment | coefficient | coefficient |
| H soil | Control | 0.722 | 0.054 |
| | group | | |
| | NJ20 | 0.574** | 0.053 |
| F soil | Control | 0.170 | 0.129 |
| | group | | |
| | NJ20 | 0.120** | 0.124* |

Note:
*represents 0.05,
**represents p < 0.01, indicating that there is significant difference between treatments.

It can be seen from Table 4 and Table 5 that in the two test soils, the strain NJ20 can reduce the enrichment coefficient of Cd and Cd transport coefficient of roots-straws. In the F soil, the strain NJ20 can reduce the enrichment coefficient of Pb. In the two test soils, the strain NJ20 can reduce the transport coefficient of Pb from roots to straws and roots to grains.

### Example 8: Effect of Strain NJ20 on Organic Matters and Available Phosphorus in Soil

Test plant rhizosphere soil was collected from example 7.

Determination method of organic matters in soil: Soil sample was dried in air and sieved, 0.200 g of rhizosphere soil was weighed, 10 mL of 0.136 mol/L K₂Cr₂O₇—H₂SO₄ standard solution was added, and the above materials were evenly mixed by gently shaking; the mixed solution was heated at 170-190° C., and 7 min was timed when condensate water occurs in the tube. After cooling, 3-4 drops of o-phenanthroline indicator were added, then titering was performed using a 0.2 mol/L ferrous sulfate standard solution, the color of the solution turned green from yellow and then suddenly turned into reddish brown, and titering endpoint was achieved.

The content of available phosphorous in soil was determined using a molybdenum antimony colorimetric method.

The content of organic matters in soil is an important indicator for evaluating the fertility of soil. It can be seen from Table 6 that in the two soil pot experiments, compared with control group without inoculation of strain NJ20, the inoculation of strain NJ20 can greatly increase the contents of organic matters in soil by 10.30%-16.94%. In addition, the inoculation of strain NJ20 can also significantly increase the content of available phosphorus in soil, which plays an important role in improving phosphorus nutrition in plants.

TABLE 6
Effect of strain NJ20 on contents of organic
matters and available phosphorus in soil

| | | Organic matter | Content of available |
|---|---|---|---|
| Soil | Treatment | (g/kg) | phosphorus (mg/kg) |
| H soil | Control group | 23.50 ± 0.69 | 61.80 ± 0.45 |
| | NJ20 | 27.48 ± 0.00* | 63.71 ± 0.67* |
| F soil | Control group | 15.53 ± 0.01 | 26.55 ± 0.40 |
| | NJ20 | 17.13 ± 0.69* | 35.30 ± 8.81* |

In summary, the screened strain NJ20 in the present disclosure can grow well under the stress of lead and cadmium, and remove soluble lead and cadmium in a culture solution by adsorption; meanwhile the strain NJ20 can generate indoleacetic acid, and has a good dissolution effect on insoluble tricalcium phosphate and improves phosphorus nutrition in soil; the inoculation of the strain of the present disclosure in heavy metal contaminated soil can effectively reduce uptake of heavy metal lead and cadmium by wheat, reduce the transport coefficient of heavy metals, and decrease the contents of lead and cadmium in wheat grains and straws.

The above descriptions are only preferred embodiments of the present disclosure, but not intended to limit the present disclosure. Any modifications, equivalent replacements and improvements made within the spirit and principle of the present disclosure should be included within the scope of protection of the present disclosure.

What is claimed is:

1. Agrobacterium for reducing uptake of heavy metals by wheat, wherein the Agrobacterium is strain NJ20, which is identified as Agrobacterium sp. and deposited in China General Microbiological Culture Collection Center under Accession CGMCC No. 26197.

2. A use of the Agrobacterium for reducing uptake of heavy metals by wheat according to claim 1 for reducing uptake of lead and cadmium by wheat in heavy metal contaminated soil.

3. A bacterial agent prepared by using the Agrobacterium for reducing uptake of heavy metals in heavy metal soil by wheat according to claim 1.

4. The bacterial agent according to claim 3, wherein the bacterial agent is a bioremediation liquid bacterial agent containing strain NJ20 whose effective viable count is greater than 200 million CFU/milliliter.

5. A use of the bacterial agent according to claim 3 for reducing uptake of lead and cadmium in heavy metal contaminated soil by wheat.
